(19) United States
(12) Patent Application Publication
(10) Pub. No.: US 2020/0261104 A1
(43) Pub. Date: Aug. 20, 2020

(54) MEDICAL GRIPPING TOOL

(71) Applicant: OLYMPUS CORPORATION, Tokyo (JP)

(72) Inventors: Shuya JOGASAKI, Tokyo (JP); Noriaki YAMANAKA, Tokyo (JP)

(73) Assignee: OLYMPUS CORPORATION, Tokyo (JP)

(21) Appl. No.: 16/866,795

(22) Filed: May 5, 2020

Related U.S. Application Data
(63) Continuation of application No. PCT/JP2017/040840, filed on Nov. 14, 2017.

(51) Int. Cl. A61B 17/29; A61B 17/062; A61B 17/04

(52) U.S. Cl. CPC A61B 17/29; A61B 2017/2939; A61B 17/0469; A61B 17/062

(57) ABSTRACT

A medical gripping tool includes: an elongated insertion section; a gripping section; an operating unit; and a wire that transmits, to the gripping section, a driving force input to the operating unit. The gripping section includes a pair of gripping pieces supported so as to be relatively swivelable. The pair of gripping pieces include a pair of first gripping sections, and a pair of second gripping sections that are disposed at positions farther away from a swiveling axis than the pair of first gripping sections are. In a state in which the pair of gripping pieces are closed by the driving force transmitted by the wire, opposed surfaces of the pair of first gripping sections are disposed with a first distance therebetween, and opposed surfaces of the pair of second gripping sections are disposed with a second distance, which is longer than the first distance, therebetween.

### CROSS-REFERENCE TO RELATED APPLICATIONS

This is a continuation of International Application PCT/JP2017/040840 which is hereby incorporated by reference herein in its entirety.

### TECHNICAL FIELD

The present invention relates to a medical gripping tool.

### BACKGROUND ART

When tissue is to be stitched under endoscopic examination, a tissue gripping tool for gripping the tissue and a needle gripping tool for gripping a needle are introduced through a channel of an endoscope or an overtube. In this case, in order to stitch the tissue, the tissue is gripped by the tissue gripping tool and is then punctured with the needle gripped by the needle gripping tool (refer to, for example, PTL 1).

### CITATION LIST

### Patent Literature

### {PTL 1}

Publication of Japanese Patent No. 3625894

### SUMMARY OF INVENTION

One aspect of the present invention is a medical gripping tool including: an elongated insertion section; a gripping section disposed at a distal end of the insertion section; an operating unit provided at a proximal end of the insertion section; and a wire that transmits, to the gripping section, a driving force input to the operating unit, wherein the gripping section includes a pair of gripping pieces supported so as to be relatively swivelable, the pair of gripping pieces include a pair of first gripping sections, and a pair of second gripping sections that are disposed at positions farther away from a swiveling axis than the pair of first gripping sections are, and in a state in which the pair of gripping pieces are closed by the driving force transmitted by the wire, opposed surfaces of the pair of first gripping sections are disposed with a first distance therebetween, and opposed surfaces of the pair of second gripping sections are disposed with a second distance therebetween, the second distance being longer than the first distance.

### BRIEF DESCRIPTION OF DRAWINGS

FIG. 1 is a side elevational view showing a medical gripping tool according to one embodiment of the present invention.

FIG. 2 is a side elevational view showing a gripping section in a closed state, disposed at the distal end of the medical gripping tool in FIG. 1.

FIG. 3 is a side elevational view showing a case where the gripping section in FIG. 2 is in an opened state.

FIG. 4 is a perspective view partially showing a first gripping piece or a second gripping piece that constitute the gripping section in FIG. 2.

FIG. 5 is a perspective view showing a modification of the first gripping piece and the second gripping piece in FIG. 4.

FIG. 6 is a side elevational view showing a modification of the gripping section in FIG. 2.

FIG. 7 is a side elevational view showing a case where the gripping section in FIG. 6 is in an opened state.

FIG. 8 is a transverse section showing a state in which a curved needle is gripped by the needle gripping sections of the gripping section in FIG. 6.

FIG. 9 is a transverse section showing a state in which a straight needle is gripped by the needle gripping sections of the gripping section in FIG. 6.

FIG. 10 is another modification of the gripping section in FIG. 2 and is a transverse section showing a state in which a curved needle is gripped by the needle gripping sections.

FIG. 11 is another modification of the gripping section in FIG. 2 and is a transverse section showing a state in which a needle is gripped by the needle gripping sections.

FIG. 12 is a side elevational view showing a gripping section provided with needle gripping sections similar to those shown in FIG. 11.

FIG. 13 is a perspective view showing another modification of the first gripping piece and the second gripping piece in FIG. 4.

FIG. 14 is a graph showing how the gripping pressure applied by the gripping section in FIG. 13 changes depending on the position in the radial direction centered on the swiveling axial line.

FIG. 15 a modification of the gripping section in FIG. 2 and is a side elevational view showing a state in which the tissue gripping sections are slowly opened and closed.

FIG. 16 shows the gripping section in FIG. 15 and is a side elevational view showing a state in which the needle gripping sections are quickly opened and closed.

FIG. 17 shows a modification of the gripping section in FIG. 2 and is a side elevational view showing a gripping section having tilted surfaces that facilitate the introduction of a needle into the needle gripping sections.

FIG. 18 shows a modification of the gripping section in FIG. 2 and is a side elevational view showing a gripping section having a magnet that facilitates the introduction of a needle into the needle gripping sections.

### DESCRIPTION OF EMBODIMENTS

A medical gripping tool 1 according to one embodiment of the present invention will now be described with reference to the drawings.

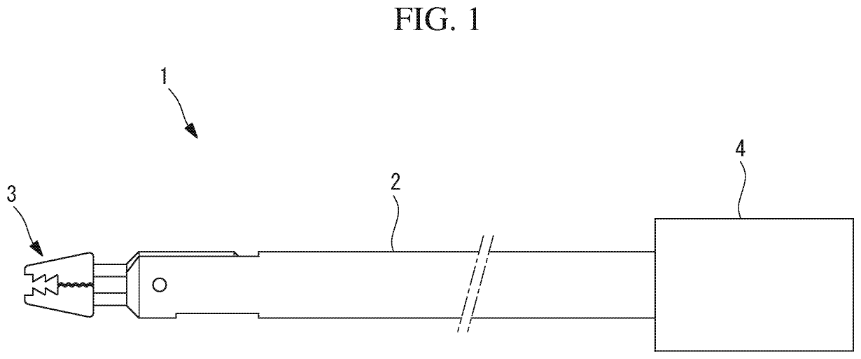

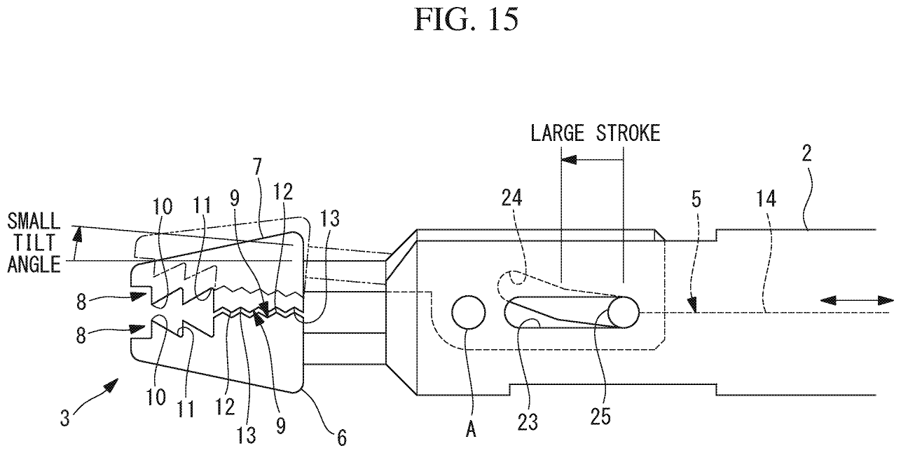

As shown in FIG. 1, the medical gripping tool 1 according to this embodiment includes: an elongated, soft insertion section 2; a gripping section 3 disposed at the distal end of the insertion section 2; an operating unit 4 disposed at the proximal end of the insertion section 2; and a force transmission unit (refer to FIG. 15) 5 for transmitting, to the gripping section 3, a driving force applied at the operating unit 4.

The insertion section 2 is a tube having at least one lumen passing therethrough in the longitudinal direction. When the insertion section 2 is inserted into a body cavity, the insertion section is bent in conformance with the shape of the body cavity as a result of being inserted through a channel in an endoscope or an overtube, thereby making it possible to place the gripping section 3 at the distal end of the insertion section 2 in the vicinity of an affected area.

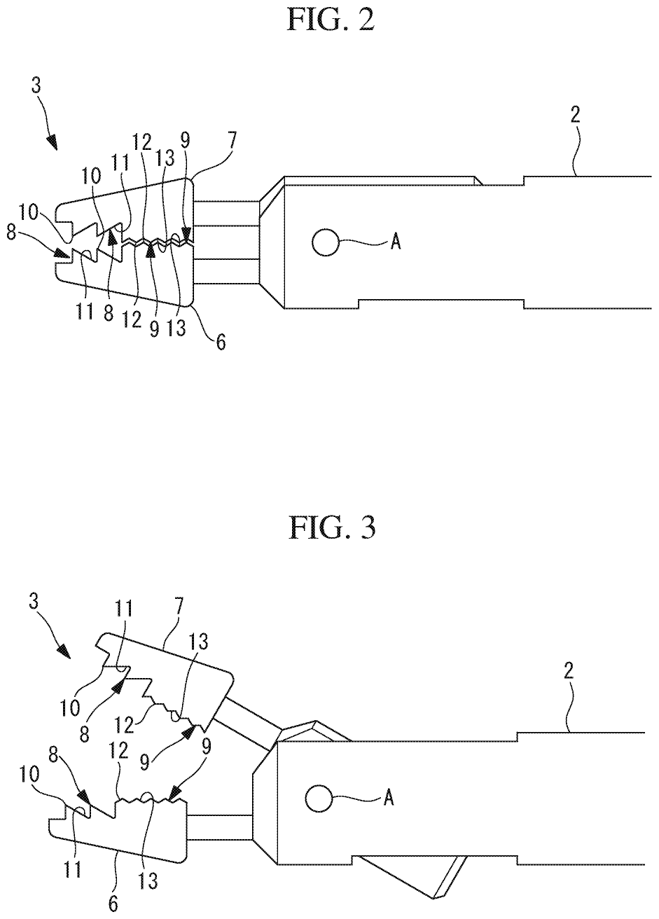

As shown in FIGS. 2 and 3, the gripping section 3 includes: a first gripping piece (gripping piece) 6 fixed to the distal end of the insertion section 2; and a second gripping piece (gripping piece) 7 supported so as to be swivelable relative to the first gripping piece 6 about a swiveling axial line (swiveling axis) A orthogonal to the longitudinal axis of the insertion section 2. As a result of the second gripping piece 7 being swiveled about the swiveling axial line A, these gripping pieces 6 and 7, constituting a pair, can be opened/closed relative to each other between a closed state in which the distal ends thereof are brought closer to each other, as shown in FIG. 2, and an opened state in which the distal ends thereof are moved away from each other, as shown in FIG. 3.

Each of the first gripping piece 6 and the second gripping piece 7 includes a tissue gripping section (second gripping section) 8 and a needle gripping section (first gripping section) 9. The tissue gripping sections 8 and the needle gripping sections 9 are provided on the opposed surfaces, i.e., the surfaces facing each other, of the first gripping piece 6 and the second gripping piece 7.

The tissue gripping sections 8 are arranged on the distal end sides of the first gripping piece 6 and the second gripping piece 7, i.e., the distal end sides that are away from the swiveling axial line A. The needle gripping sections 9 are arranged adjacent to the proximal end sides of the tissue gripping sections 8.

The tissue gripping sections 8 include first protruding sections 10 and first recessed sections 11 provided on the opposed surfaces that are disposed in a manner sufficiently spaced apart from each other even in the closed state shown in FIG. 2.

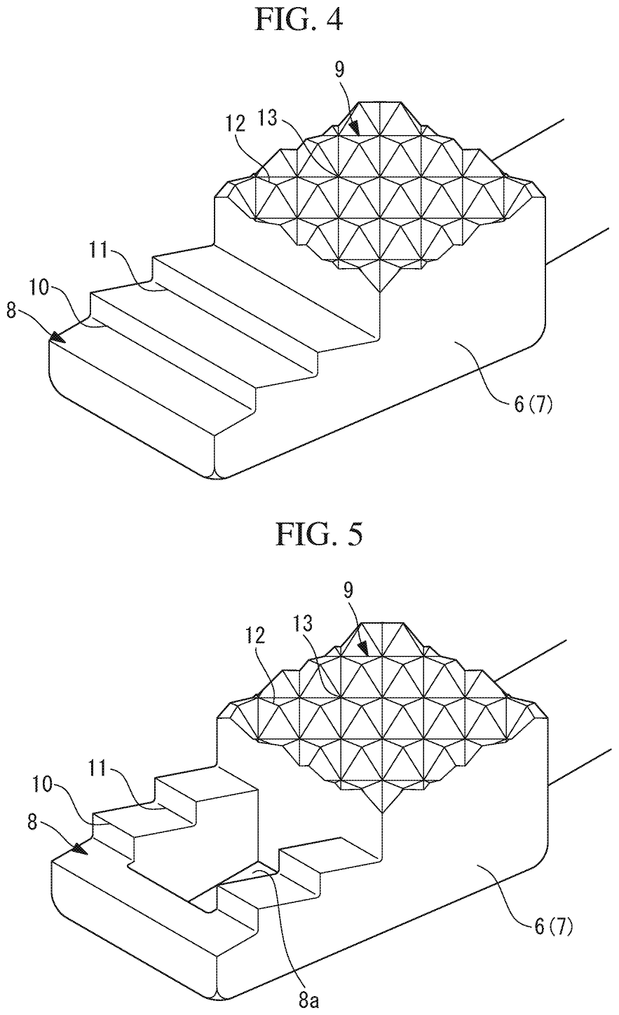

As shown in FIG. 4, the first protruding sections 10 and the first recessed sections 11 extend in a straight line on the opposed surfaces of the first gripping piece 6 and the second gripping piece 7 over the entire widthwise length and are alternately arranged in a radial direction centered on the swiveling axial line A.

As shown in FIG. 2, the first protruding sections 10 and the first recessed sections 11 of the first gripping piece 6 and the second gripping piece 7 are arranged so that in the closed state, the first protruding sections 10 face each other and the first recessed sections 11 face each other.

In addition, the needle gripping sections 9 include second protruding sections 12 and second recessed sections 13 provided on the opposed surfaces that are arranged sufficiently adjacent to each other in the closed state shown in FIG. 2.

As shown in, for example, FIG. 4, the second protruding sections 12 and the second recessed sections 13 are alternately arranged in a radial direction centered on the swiveling axial line A and in the width direction orthogonal to the radial direction by arranging quadrangular pyramids of the same shape in rows and columns on a flat surface.

In the needle gripping sections 9, the second protruding sections 12 and the second recessed sections 13 of the first gripping piece 6 and the second gripping piece 7 are arranged so that each of the second protruding sections 12 faces one of the second recessed sections 13 in the closed state shown in FIG. 2. By doing so, in the needle gripping sections 9, the second protruding sections 12 mesh with the second recessed sections 13 in the closed state, as shown in FIG. 2.

The operating unit 4 has an arbitrary known structure operated by an operator and is configured to supply, as a force along the longitudinal direction of the insertion section 2, the force applied by the operator to the force transmission unit 5.

The force transmission unit 5 includes a wire (refer to FIG. 15) 14 that passes through the lumen of the insertion section 2 and that links the operating unit 4 to the gripping section 3. The force transmission unit 5 is configured to transmit, as a tensile force, the force supplied at the operating unit 4 to the gripping section 3.

When the wire 14 is pulled towards the proximal end side, the second gripping piece 7 is swiveled relative to the first gripping piece 6 about the swiveling axial line A by means of the tensile force transmitted by the wire 14.

The operation of the medical gripping tool 1 according to this embodiment with the above-described structure will be described below.

In order to treat an affected area by using the medical gripping tool 1 according to this embodiment, the medical gripping tool 1 is inserted into a channel of the endoscope or the overtube having been inserted into the body in a state in which the second gripping piece 7 is closed relative to the first gripping piece 6 as shown in FIG. 2, is projected from the opening at the distal end of the channel, and is then disposed so as to face the affected area.

In this state, the operator applies a force for pushing the wire 14 towards the distal end side by operating the operating unit 4, thereby swiveling the second gripping piece 7 relative to the first gripping piece 6 until the opened state as shown in FIG. 3 is achieved.

Thereafter, the tissue to be gripped is disposed between the tissue gripping sections 8 provided at the distal end sections of the first gripping piece 6 and the second gripping piece 7, and the wire 14 is then pulled towards the proximal end side by operating the operating unit 4. By doing so, the second gripping piece 7 can be rotated relative to the first gripping piece 6 about the swiveling axial line A. This allows the gripping section 3 to be placed in the closed state as shown in FIG. 2, thereby making it possible to grip the tissue between the tissue gripping sections 8.

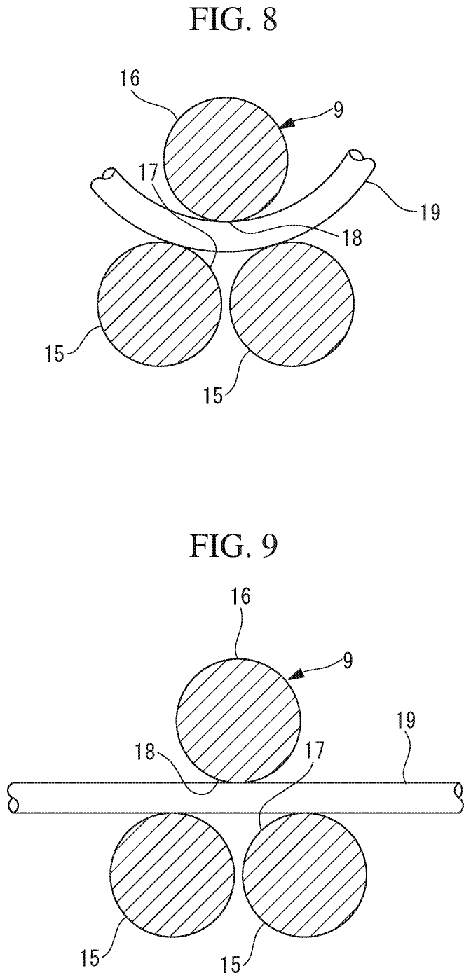

Meanwhile, a needle (refer to FIG. 8) 19 to be gripped is disposed between the needle gripping sections 9 arranged adjacent to the proximal end sides of the tissue gripping sections 8 of the first gripping piece 6 and the second gripping piece 7, and the wire 14 is then pulled towards the proximal end side by operating the operating unit 4. By doing so, the second gripping piece 7 can be rotated relative to the first gripping piece 6 about the swiveling axial line A. This allows the gripping section 3 to be placed in the closed state as shown in FIG. 2, thereby making it possible to grip the needle 19 between the needle gripping sections 9.

In this case, according to the medical gripping tool 1 of this embodiment, a sufficiently large gap (second distance) is formed between the opposed surfaces of the tissue gripping sections 8 even in the case where the gripping section 3 is in the closed state, as shown in FIG. 2, thus softly biting the tissue to be gripped. Because of this, the tissue is subjected to less damage and is maintained in a healthy state. In addition, because the first protruding sections 10 on the opposed surfaces face each other and the first recessed sections 11 on the opposed surfaces face each other in the closed state, the gap formed between the opposed surfaces becomes small at positions at which the first protruding sections 10 face each other, whereas the gap formed between the opposed surfaces becomes large at positions at which the first recessed sections 11 face each other.

Therefore, the tissue pinched between the tissue gripping sections 8 can be gripped so firmly as not to fall because the tissue is bitten by the first protruding sections 10. Still, it is possible to more reliably prevent the tissue from being crushed due to gripping because the gripped tissue is pinched only softly in the wide gap and escapes into the wide gap between the first recessed sections 11. In short, the present invention affords an advantage in that tissue can be gripped so firmly as not to fall without being crushed.

On the other hand, in the needle gripping sections 9, as a result of the second protruding sections 12 on the opposed surfaces meshing with the second recessed sections 13 on the opposed surfaces, the gaps (first distance) between the second protruding sections 12 and the second recessed sections 13 are small. Therefore, it is possible to prevent falling of the needle 19 by pinching the needle 19 with a large gripping force. In particular, because the needle gripping sections 9 are disposed closer to the proximal end side in the radial direction than the tissue gripping sections 8 are, the needle gripping sections 9 can generate a larger gripping force than the tissue gripping sections 8 when the same operating force is applied by means of the operating unit 4. In short, the present invention has an advantage in that the needle 19, which is not damaged even when a large force is applied thereto, can be firmly gripped with the maximum possible gripping force.

Also, the present invention affords an advantage in that because the medical gripping tool 1 according to this embodiment includes both the tissue gripping sections 8 and the needle gripping sections 9, tissue can be handled seamlessly, i.e., without having to replace the medical gripping tool 1 by removing/inserting the medical gripping tool 1 from/into a channel, when tissue having been gripped and treated is to be stitched by gripping the needle 19. This allows easy treatment. There is another advantage in that because many sets of tissue gripping tools and needle gripping tools are not used, it is possible to prevent an increase in the diameter of the endoscope or the overtube, lessening discomfort to the patient.

Note that in this embodiment, recessed sections 8a or through-holes may be formed in the regions constituting the tissue gripping sections 8 of the first gripping piece 6 and the second gripping piece 7, as shown in FIG. 5. By doing so, it is possible to form a space for accommodating the tissue gripped by the tissue gripping sections 8, thereby keeping the tissue healthy without crushing the tissue while still firmly gripping the tissue.

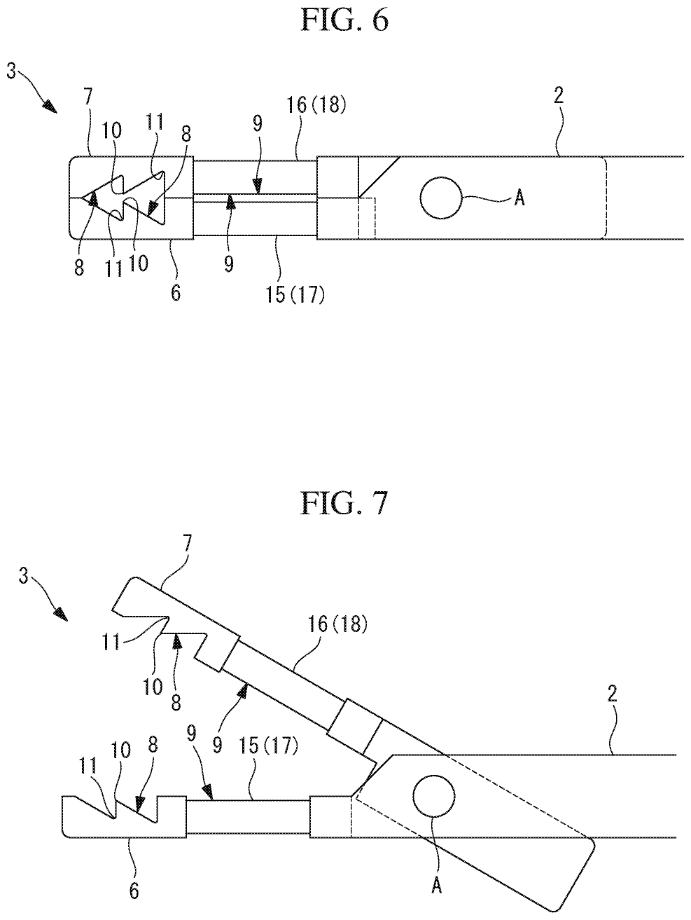

In addition, as shown in FIGS. 6 to 8, the needle gripping sections 9 may include: two columnar parts (first columnar parts and first contact section) 15 that are provided on the first gripping piece 6 and that extend in a radial direction; and one columnar part (second columnar part and second contact section) 16 that is provided on the second gripping piece 7 and that extends in a radial direction. By doing so, a second recessed section 17 formed between the two columnar parts 15 of the first gripping piece 6 is also arranged so as to mesh with a second protruding section 18 formed by the one columnar part 16 of the second gripping piece 7, thereby allowing the needle 19 to be gripped by the three columnar parts 15 and 16 at three locations along the longitudinal direction of the needle 19, as shown in FIG. 8.

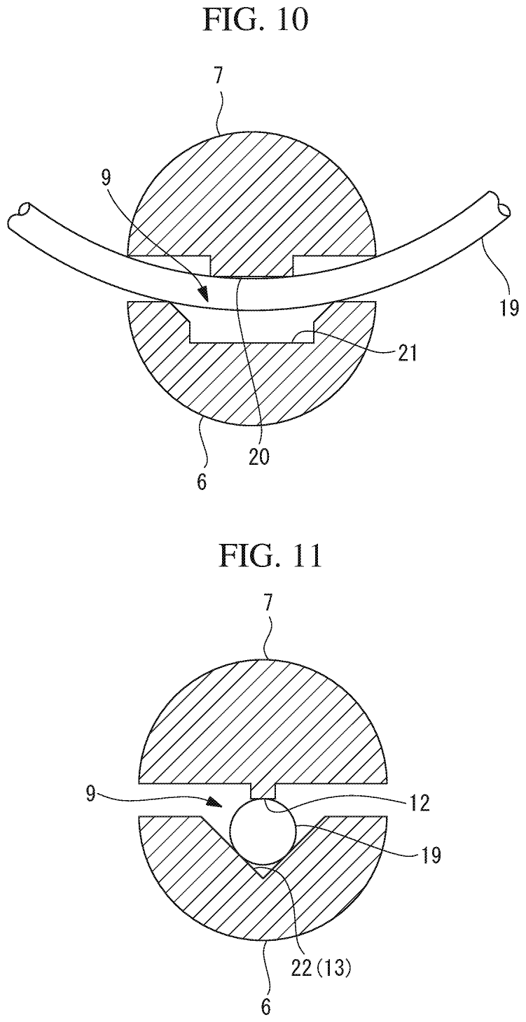

With this configuration, not only a curved needle 19 as shown in FIG. 8 but also a straight needle 19 as shown in FIG. 9 can be firmly gripped at three locations thereof by adjusting the relative swiveling angle between the first gripping piece 6 and the second gripping piece 7.

In contrast to the above, the first gripping piece 6 may be provided with one columnar part 16, and the second gripping piece 7 may be provided with two columnar parts 15.

In addition, as shown in FIG. 10, the needle gripping sections 9 may be configured from a second recessed section 20 formed in one of the first gripping piece 6 and the second gripping piece 7 and a second protruding section 21 formed on the other. In the same manner as in FIGS. 8 and 9, this also allows the needle gripping sections 9 to firmly grip the needle 19 disposed in the width direction of the needle gripping sections 9 such that the needle gripping sections 9 are in contact with the needle 19 at three longitudinal locations thereof.

A V-shaped groove 22 as shown in FIGS. 11 and 12 may be employed as a second recessed section 13. If the first gripping piece 6 is provided with the V-shaped groove (first groove section) 22 along a radial direction centered on the swiveling axial line A, as shown in, for example, FIG. 11, a second protruding section 12 can be provided at a position in the second gripping piece 7, i.e., the position facing the V-shaped groove 22, thereby allowing the needle 19 to be gripped at three locations spaced apart in the circumferential direction of a transverse section of the needle 19 arranged along the V-shaped groove 22.

Also in the case where the V-shaped groove (second groove section) 22 is provided along the width direction of the needle gripping sections 9 as shown in FIG. 12, the needle 19 disposed along the width direction of the needle gripping sections 9 can be firmly griped. In addition, a V-shaped groove (first groove section) 22 may be provided along the width direction of the needle gripping sections 9, and another V-shaped groove (second groove section) 22 may be provided along the width direction of the needle gripping sections 9.

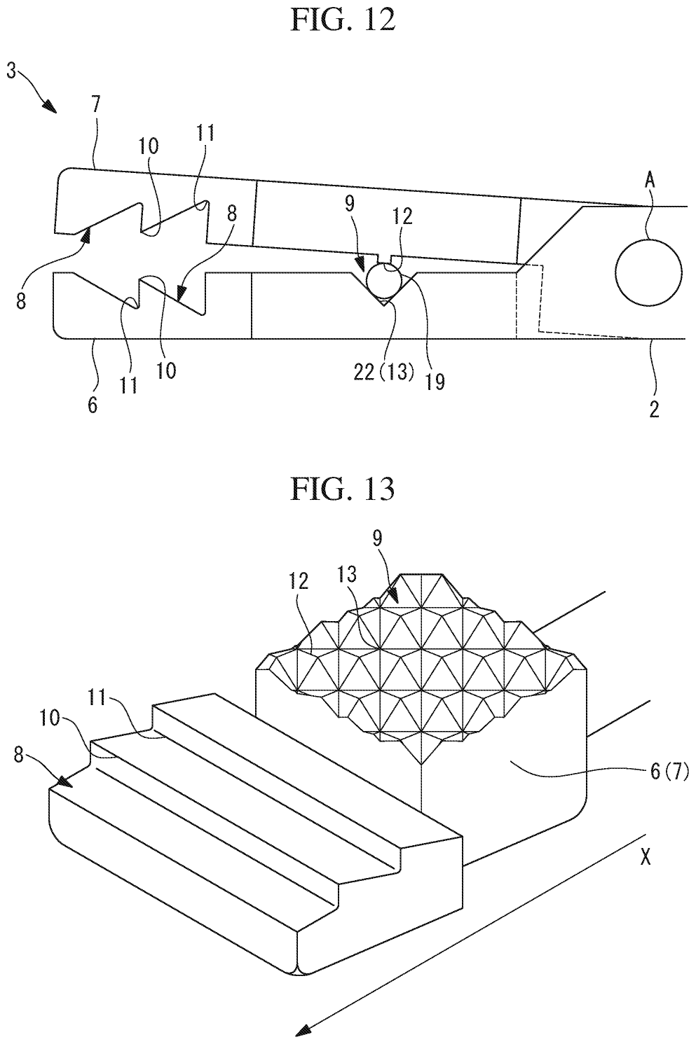

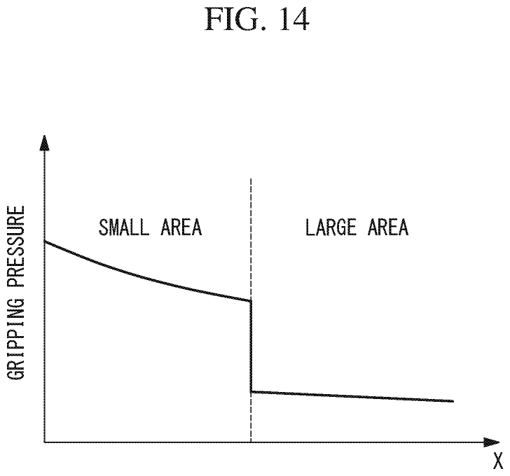

In addition, as shown in FIG. 13, the needle gripping sections 9 and the tissue gripping sections 8 may be made to have different widths from each other. In the example shown in FIG. 13, the area of each of the tissue gripping sections 8 can be easily increased by making the width of the tissue gripping section 8 larger than the width of each of the needle gripping sections 9, thereby affording an advantage in that the gripping pressure acting on the pinched tissue can be significantly decreased, as shown in FIG. 14.

In addition, as shown in FIG. 13, steps can be formed between the tissue gripping section 8 and the needle gripping section 9 by making the width of the tissue gripping section 8 sharply different from the width of the needle gripping section 9. This allows the curved needle 19 to be easily positioned by abutting, against the steps, the curved needle 19 gripped by the needle gripping sections 9. In addition, providing the steps allows the tissue gripping sections 8 to be easily recognized as a result of being discriminated from the needle gripping sections 9 from outside. By doing so, tissue can be handled in a protected manner by reliably gripping the tissue by means of the tissue gripping sections 8 under endoscopic examination, and furthermore, the tissue can be stitched with the needle 19 with a decreased risk of dropping the needle 19 by reliably gripping the needle 19 by means of the needle gripping sections 9.

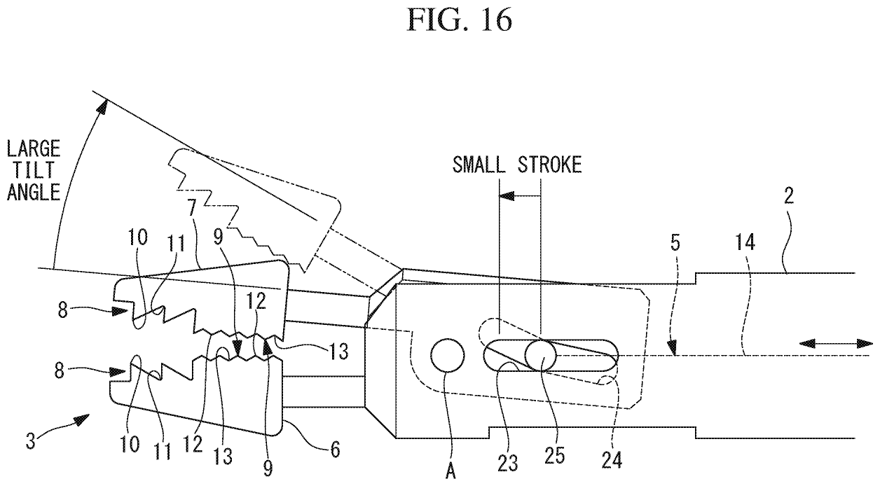

In addition, because the tissue gripping sections 8 are disposed on the distal end sides of the first gripping piece 6 and the second gripping piece 7, and the needle gripping sections 9 are disposed on the proximal end sides of the first gripping piece 6 and the second gripping piece 7, a sufficient space between the first gripping piece 6 and the second gripping piece 7 cannot be secured at the needle gripping sections 9 without swiveling the second gripping piece 7 by a large amount relative to the first gripping piece 6. To address this issue, a configuration as shown in FIGS. 15 and 16 may be employed, wherein the first gripping piece 6 is provided with a first slit 23 extending in the longitudinal direction of the insertion section 2, the second gripping piece 7 is provided with a second slit 24 intersecting the first slit 23, and a pin 25 that is accommodated at the intersection of the first slit 23 and the second slit 24 is pushed and pulled with the wire 14 constituting the force transmission unit 5.

In this case, the present invention may be configured such that the second slit 24 is in a shape that is curved at an intermediate point thereof as shown in FIGS. 15 and 16. By doing so, even a large stroke of the pin 25 causes the second gripping piece 7 to be opened only slightly, as shown in FIG. 15, while the open angle of the second gripping piece 7 relative to the first gripping piece 6 is from the angle in the closed state to a first angle, which is larger than the angle in the closed state but smaller than the angle in the opened state. On the other hand, even a small stroke of the pin 25 causes the second gripping piece 7 to be opened by a large amount, as shown in FIG. 16, while the open angle of the second gripping piece 7 is from the first angle to the angle in the opened state.

In short, it is possible to prevent tissue from being abruptly pressed because the second gripping piece 7 is slowly opened and closed until the first angle is reached. On the other hand, it possible to enhance the operability when the needle 19 is to be gripped and mounted because the second gripping piece 7 is quickly opened and closed between the first angle and the angle in the opened state.

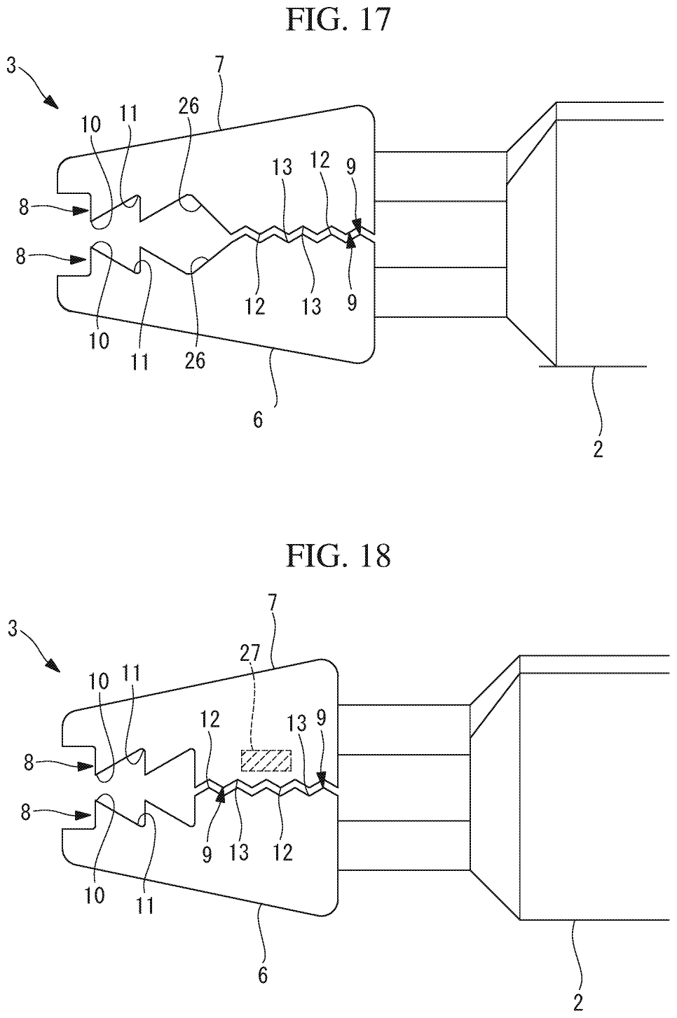

In addition, in order to easily mount the needle 19 on the needle gripping sections 9, tilted surfaces 26 tilting from the tissue gripping sections 8 towards the needle gripping sections 9 may be provided as shown in FIG. 17, so that the needle 19 can be mounted while being guided along the tilted surfaces 26. Alternatively, round surfaces may be provided instead of the tilted surfaces 26.

In addition, the first gripping piece 6 and the second gripping piece 7 may be formed of a non-magnetic material, and a magnet 27 may be embedded at the position corresponding to the needle gripping section 9 of at least one of the first gripping piece 6 and the second gripping piece 7, as shown in FIG. 18.

By doing so, the work of guiding the needle 19 to the needle gripping sections 9 can be made easier by attracting the needle 19 formed of a magnetic material with the magnetism attractive force of the magnet 27.

In addition, although this embodiment has been described by way of an example where the elongated insertion section 2 is soft, instead of this, a rigid insertion section 2 may be employed.

The above-described embodiment also leads to the following aspects.

One aspect of the present invention is a medical gripping tool including: an elongated insertion section; a gripping section disposed at a distal end of the insertion section; an operating unit provided at a proximal end of the insertion section; and a force transmission unit that transmits, to the gripping section, a driving force input to the operating unit, wherein the gripping section includes a pair of gripping pieces supported so as to be relatively swivelable, and the pair of the gripping pieces include a pair of needle gripping sections that grip a needle and a pair of tissue gripping sections that are disposed at positions farther away from a swiveling axis than the pair of needle gripping sections are and that grip tissue.

According to this aspect, when an operator operates the operating unit on the proximal end side of the insertion section and inputs a driving force, the input driving force is transmitted to the distal end of the insertion section by means of the force transmission unit, thereby relatively swiveling the pair of gripping pieces of the gripping section. When tissue is to be gripped, the tissue can be pinched and gripped by means of the tissue gripping sections disposed at a position away from the swiveling axis of the gripping pieces. When a needle is to be gripped, the needle can be pinched and gripped by means of the needle gripping sections disposed at a position near the swiveling axis of the gripping section.

This eliminates the necessity to replace the medical gripping tool by removing/inserting the medical gripping tool from/into a channel, thus enabling smooth treatment such that the needle is gripped by means of the same medical gripping tool that has been used to grip the tissue, or the tissue is gripped by means of the same medical gripping tool that has been used to grip the needle. In addition, it is not necessary to arrange many pairs of tissue gripping tools and needle gripping tools simultaneously, thereby preventing an increase in the diameter of the endoscope or the overtube. In addition, the needle in the needle gripping sections, which are disposed at positions near the swiveling axis, can be firmly gripped with a gripping force that is larger than in the tissue gripping sections, which are disposed at positions away from the swiveling axis.

In the above-described aspect, the pair of tissue gripping sections may include first recessed sections and first protruding sections that are formed on opposed surfaces of the pair of gripping pieces, and in a state in which the pair of gripping pieces are closed, the first recessed sections on the opposed surfaces of the pair of gripping pieces may be disposed at mutually facing positions, and the first protruding sections on the opposed surfaces of the pair of gripping pieces may be disposed at mutually facing positions.

By doing so, even when the pair of gripping pieces are closed to the maximum possible extent, the mutually facing first protruding sections provided on the opposed surfaces come close to each other, and a relatively large gap is formed between the mutually facing first recessed sections provided on the opposed surfaces. As a result, when the tissue is disposed and pinched between the opposed surfaces, the tissue can be accommodated in the gap formed between the first recessed sections, making it possible to firmly grip the tissue without crushing a large area of the tissue.

In addition, in the above-described aspect, the pair of needle gripping sections may include second recessed sections and second protruding sections that are formed on the opposed surfaces of the pair of gripping pieces, and in a state in which the pair of gripping pieces are closed, the second recessed sections and the second protruding sections on the opposed surfaces of the pair of gripping pieces may be disposed at positions at which the second recessed sections mesh with the second protruding sections.

By doing so, when the pair of gripping pieces are closed, the second recessed sections provided on the opposed surfaces mesh with the second protruding sections provided on the opposed surfaces, making it possible to more reliably grip the needle in a small gap between the opposed surfaces.

In addition, in the above-described aspect, the pair of needle gripping sections may include, in one of the pair of gripping pieces, first contact section coming into contact with a needle at two locations on an outer circumferential surface thereof and may include, in the other of the pair of gripping pieces, a second contact section coming into contact with the needle at one location on the outer circumferential surface thereof.

By doing so, when the needle is disposed between the opposed surfaces of the needle gripping sections, the needle can be stably gripped because the needle is gripped at a total of three locations on the outer circumferential surface thereof, i.e., the three locations including two locations in the first contact section and one location in the second contact section.

In addition, in the above-described aspect, the first contact section may include two first columnar parts that are disposed parallel to each other, and the second contact section may include a second columnar part that is disposed so as to be substantially equally spaced apart from the two first columnar parts in a radial direction.

By doing so, when the needle is to be disposed between the opposed surfaces of the needle gripping sections such that the longitudinal axis of the needle is along the direction in which the first columnar parts are arranged, the needle comes into contact with the first columnar parts at two longitudinal locations and comes into contact with the second columnar part at one radial location. Because of this, the needle can be stably gripped.

In addition, in the above-described aspect, the first contact section may include a V-shaped groove coming into contact with the needle at two locations in a circumferential direction thereof.

By doing so, when the needle is to be disposed between the opposed surfaces of the needle gripping sections such that the longitudinal axis of the needle is along the V-shaped groove provided in one of the gripping pieces, the needle comes into contact with the V-shaped groove at two locations in the circumferential direction thereof and is pressed by the other gripping piece at one location, allowing the needle to be gripped at three locations in the circumferential direction thereof. Because of this, the needle can be stably gripped.

In addition, in the above-described aspect, the V-shaped groove may include a first groove section extending along a plane that intersects the swiveling axis and a second groove section extending parallel to the swiveling axis.

By doing so, when the needle is to be disposed between the opposed surfaces of the needle gripping sections, the needle can be stably gripped in one of the longitudinal direction and the width direction of the gripping pieces by selecting one of the first groove section extending along a plane intersecting the swiveling axis and the second groove section extending so as to be parallel to the swiveling axis.

In addition, in the above-described aspect, the pair of tissue gripping sections may have a larger dimension than the pair of needle gripping sections in a direction parallel to the swiveling axis.

By doing so, because steps are formed between the tissue gripping sections and the needle gripping sections, it is possible to easily discriminate from outside between the tissue gripping sections and the needle gripping sections.

### REFERENCE SIGNS LIST

1 Medical gripping tool
2 Insertion section
3 Gripping section
4 Operating unit
5 Force transmission unit
6 First gripping piece (gripping piece)
7 Second gripping piece (gripping piece)
8 Tissue gripping section
9 Needle gripping section
10 First protruding section
11 First recessed section
12, 18, 21 Second protruding section
13, 17, 20 Second recessed section
15 Columnar part (first columnar part, first contact section)
16 Columnar part (second columnar part, second contact section)
19 Needle
22 V-shaped groove (first groove section, second groove section)
A Swiveling axial line (swiveling axis)

1. A medical gripping tool comprising:
an elongated insertion section;
a gripping section disposed at a distal end of the insertion section;
an operating unit provided at a proximal end of the insertion section; and
a wire that transmits, to the gripping section, a driving force input to the operating unit,
wherein the gripping section includes a pair of gripping pieces supported so as to be relatively swivelable,
the pair of gripping pieces include a pair of first gripping sections, and a pair of second gripping sections that are disposed at positions farther away from a swiveling axis than the pair of first gripping sections are, and
in a state in which the pair of gripping pieces are closed by the driving force transmitted by the wire, opposed surfaces of the pair of first gripping sections are disposed with a first distance therebetween, and opposed surfaces of the pair of second gripping sections are disposed with a second distance therebetween, the second distance being longer than the first distance.

2. The medical gripping tool according to claim 1, wherein the pair of gripping pieces include steps between the opposed surfaces of the pair of first gripping sections and the opposed surfaces of the pair of second gripping sections.

3. The medical gripping tool according to claim 2, wherein one of the pair of gripping pieces includes a slit having a shape that is curved at an intermediate point thereof.

4. The medical gripping tool according to claim 1,
wherein the pair of second gripping sections include first recessed sections and first protruding sections that are formed on opposed surfaces of the pair of gripping pieces, and
in a state in which the pair of gripping pieces are closed, the first recessed sections on the opposed surfaces of the pair of gripping pieces are disposed at mutually facing positions, and the first protruding sections on the opposed surfaces of the pair of gripping pieces are disposed at mutually facing positions.

5. The medical gripping tool according to claim 4,
wherein the pair of first gripping sections include second recessed sections and second protruding sections that are formed on the opposed surfaces of the pair of gripping pieces, and
in a state in which the pair of gripping pieces are closed, the second recessed sections and the second protruding sections on the opposed surfaces of the pair of gripping pieces are disposed at positions at which the second recessed sections mesh with the second protruding sections.

6. The medical gripping tool according to claim 5, wherein sizes of the first recessed sections and the first protruding sections are larger than sizes of the second recessed sections and the second protruding sections.

7. The medical gripping tool according to claim 1, wherein the pair of first gripping sections include, in one of the pair of first gripping sections, a first contact section coming into contact with a needle at two locations on an outer circumferential surface thereof and include, in the other of the pair of first gripping sections, a second contact section coming into contact with the needle at one location on the outer circumferential surface thereof.

8. The medical gripping tool according to claim 7,
wherein the first contact section includes two first columnar parts that are disposed parallel to each other, and
the second contact section includes a second columnar part that is disposed so as to be substantially equally spaced apart from the two first columnar parts in a radial direction.

9. The medical gripping tool according to claim 7, wherein the first contact section includes a V-shaped groove coming into contact with the needle at two locations in a circumferential direction thereof.

10. The medical gripping tool according to claim 9, wherein the V-shaped groove includes a first groove section extending along a plane that intersects the swiveling axis and a second groove section extending parallel to the swiveling axis.

11. The medical gripping tool according to claim 1, wherein the pair of second gripping sections have a larger width than the pair of first gripping sections in a direction parallel to the swiveling axis.
